(19) United States
(12) Patent Application Publication
(10) Pub. No.: US 2008/0318304 A1
(43) Pub. Date: Dec. 25, 2008

(54) Cultivation of micro-algae and application to animal feeds, environments, field crops, and waste treatment

(76) Inventors: Dudley Burton, Carmichael, CA (US); Warren Cleeland, Fresno, CA (US)

Correspondence Address: LAW OFFICES OF TIMOTHY BRISSON, 340 GRASS VALLEY HIGHWAY, AUBURN, CA, 95603, US

(21) Appl. No.: 12/001,773

(22) Filed: Dec. 11, 2007

Related U.S. Application Data
(60) Provisional application No. 60/869,478, filed on Dec. 11, 2006.

(51) Int. Cl. C12N 1/12

(52) U.S. Cl. 435/257.1

(57) ABSTRACT

The present disclosure concerns producing a micro-algae product. In preferred embodiments, the method comprises collecting urine from lactating cows, mixing the collected urine with aerobically digested cow manure to form a mother liquor, fermenting the mother liquor in an algae growth tank; and forming two distinct layers of top water, including a top water layer, the top-water layer including yeast by-products and algae by-products.

### RELATED APPLICATIONS

This application claims priority to U.S. provisional application 60/869,478, filed Dec. 11, 2006,

### FIELD OF THE DISCLOSURE

The present disclosure relates generally to the cultivation of micro-algae and its use and application for animals, especially ruminant animals.

### BACKGROUND

It is well-known in commercial practice to grow and harvest micro-algae. Some of these formulations are to improve human health; some are to reduce odor; and some are for the purposes of treating waste lagoons. However, none of these systems involve the separation and collection of “top water” as the concentrated polysaccharide by-product of the algae production. Further, none of the prior systems apply this “top water” to the feeding of ruminants, as opposed to feeding the algae directly to humans or animals.

In short, existing systems for using micro-algae in ruminant feeds and the allied uses are non-existent, not effective, or too expensive. This invention resolves these problems by providing a cost-effective, predictable, controlled system for the production and use of algal by-products to achieve substantial health and economic goals.

### BRIEF DESCRIPTION OF THE DRAWINGS

FIG. 1 is a diagram of a process for producing a micro-algae product and separating a top water component.

FIGS. 2 and 3 are conceptual diagrams of systems for the growing and production of a micro-algae product and separating a top water component.

### DETAILED DESCRIPTION

Embodiments of the present invention described in the following detailed description are directed at virtual agents. Those of ordinary skill in the art will realize that the detailed description is illustrative only and is not intended to restrict the scope of the claimed inventions in any way. Other embodiments of the present invention, beyond those embodiments described in the detailed description, will readily suggest themselves to those of ordinary skill in the art having the benefit of this disclosure. Reference will now be made in detail to implementations of the present invention as illustrated in the accompanying drawings. Where appropriate, the same reference indicators will be used throughout the drawings and the following detailed description to refer to the same or similar parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

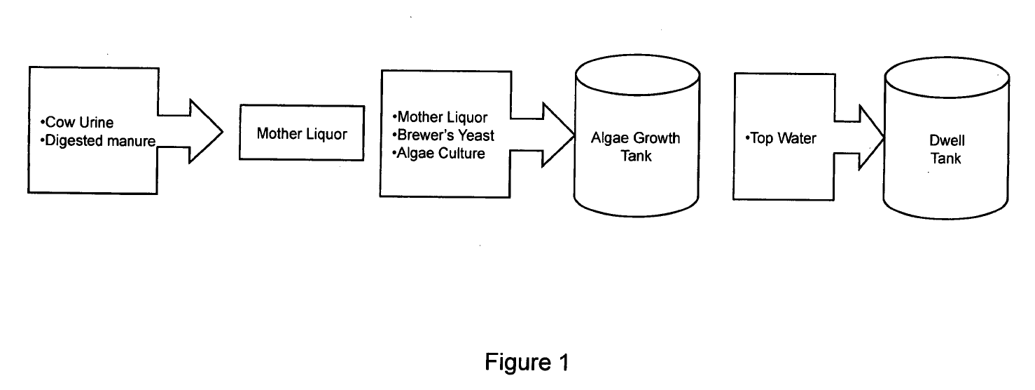

FIG. 1 is an overview of a process for producing a micro-algae product. The process begins by collecting pure urine from healthy, lactating cows. In one embodiment, the urine may be mixed with anaerobically digested cow manure to form a mother liquor. Alternatively, fresh manure may be used alone or in a mixture with anaerobically digested cow manure. In preferred embodiments, the ration of urine to manure is about a 1:1 ratio. A mixture of 1-2% brewer's yeast may be added. The mixture is then allowed to ferment in an algae growth tank. While the anaerobic digestion process eliminates any pesticide residues, it appears that hormones from the urine remain, because dairymen report better conception rates with the use of this system.

The present disclosure may relate to the formation of a “mother liquor”, such as that disclosed in U.S. Pat. No. 3,855,121 to Gough. This disclosure also may discuss discusses the physical and chemical properties of the polysaccharide-rich water such as that discussed in U.S. Pat. No. 3,958,364 to Schenck. Finally, the present disclosure may relate to alternatives for microbial and algal species such as that discussed in Haerther U.S. Pat. No. 6,896,804. Each of the foregoing is hereby incorporated by reference as thought fully set for herein.

The advantage of anaerobically digested manure is that it is pathogen free. Samples tested for e coli and salmonella were negative. In addition, the nutrients have not been in an oxidizing environment, so the oxygen-rich environment of the algae pools creates possibilities for additional chemical transformations.

In preferred embodiments, the fermentation process requires 2-3 weeks. It should be noted that longer fermentation processes may yield better results. This product of this process becomes feed for the micro-algae production system.

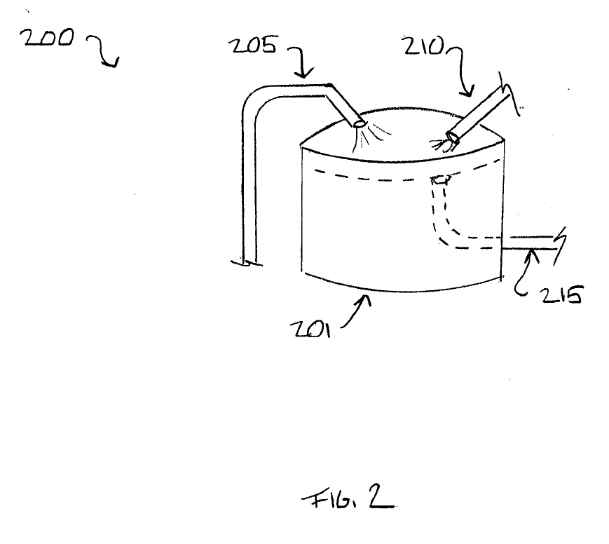

Referring now to FIG. 2, a conceptual diagram of an algae culture generation system 200 is shown. In preferred embodiments, chlorella sp. is used. It is contemplated that both chlorella (green algae) and blue-green algae (bacteria) may be employed. Strains of the chlorella and other suitable species are available from American Type Culture Collection (ATCC) and the UT Algae Collection. In one preferred embodiment, strains are produced from high nutrient ponds where cells in the air inoculate the water and start new populations, and are maintained for long periods of time.

These cultures are put into a tank 201 of water provided with spray nozzles 205 for new water supply and for providing the nutrient product. The amount of original culture used may be left to the discretion of producer. It will be understood that the less culture used, the longer it takes for the populations to multiply to equilibrium. In preferred embodiments, the water spray is designed to provide about 180 gallons per day for a 9 foot diameter pool containing about 1200 gallons of water. The water spray provides gentle circulation as well as the flow of water.

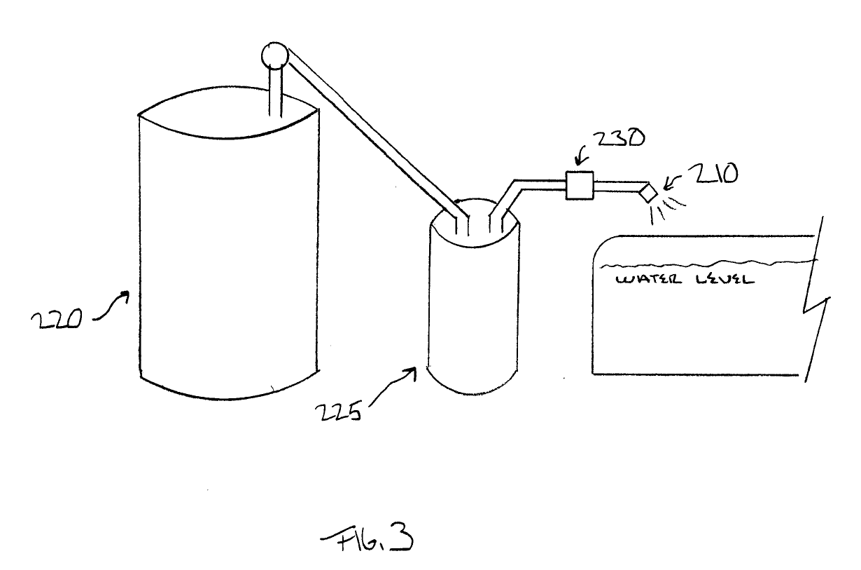

The nutrients are provided by feed sprays 210 that may include timed valves operating under pressure. Referring briefly to FIG. 3, in one embodiment, the nutrient tanks 225 that feed the feed sprays 210 are pressurized by externally provided air pressure by tank 220, and a timer 230 allows feeding of nutrients intermittently as desired. In one embodiment, the nutrients are introduced into the mixture at a rate of approximately a few ounces per day. The optimal rates of feed may be chosen based upon factors such as weather conditions, desired product throughput, and the maturity of the culture. It is contemplated that the process described herein may be highly scalable in that any number of tanks, called “generator tanks”, may be employed to provide product at any desired scale.

Referring back to FIG. 2, drains 215 are provided on the generator tank 201 to remove water from the surface of the mixture. In one embodiment, the drains 215 may comprise standpipes which are configured to remove only the topmost layer of the mixture. This arrangement enables continuous collection of “top water,” the desired algal by-product. The presence of this top water can be determined by the surface characteristics of the water, its response to oil dropped on the surface, and the popping of tiny gas bubbles as the algae produces oxygen in direct sunlight.

In preferred embodiments, two distinct layers of top water may form, each having different viscosities. For example, in an algae growth medium that is approximately 0.5 m deep, an uppermost layer of approximately 2-3 mm will form over a second layer of approximately 1 cm.

In preferred embodiments, the “top water” may contain yeast by-products and algae by-products. In addition, the polysaccharides produced by the algae may provide a matrix or tonic that supports the production of beneficial flora in the ruminant and provides significant immunological side-effects as will be more fully described below.

If necessary or desired, mosquito fish, goldfish, or koi can be put into the generator tanks to control water fleas (daphnia spp.) or other predators that prey on the algae.

Referring back to FIG. 1, the drainage top water from generator tanks is collected in a dwell tank where the aggregated top water is allowed to rest. In preferred embodiments, the period of resting may comprise approximately 24-48 hours. This process, called “dwell,” produces stronger product for use in feeds, lagoons, or fertilizers. From the dwell tank, product is collected for use.

As will now be described, the micro-algae product may be used in animal feed mixes, animal watering systems, lagoon treatments, environmental control, and field crop treatments.

In the treatment of dairy cattle, about one half gallon of product per day treats one milking cow; about one quart per day treats calves. The effect of the feed is apparent in the general appearance of the animals. These effects include shinier coats, perkier eyes and ears, and looser stools with very little undigested fiber. The polysaccharides produced by micro-algae are observed to have significant immunological, nutritional, and therapeutic benefits. Lower death rates among calves and young animals are observed, especially during the development of the rumen; improved milk production, and longer-lived animals are other benefits of the process.

In preferred embodiments, the top water may contain yeast by-products and algae by-products. Yeast and yeast by-products have many important nutritional effects. More particularly, it is known that fungi, including yeasts, produce extra-cellular enzymes, including cellulases, that help to break down fiber, making it more digestible. In addition, the polysaccharides produced by the algae may provide a matrix or tonic that supports the production of beneficial flora in the ruminant and provides significant immunological side-effects.

It is contemplated that the product of this disclosure enhances the availability of nutrients and likely producing compounds, enzymes, and associated factors that aid digestion and thereby enhance energy, stimulate beneficial processes, etc. Additionally, observed effects are often most pronounced in periods of stress—heat, calving, post-partum, weaning, etc.

The improved breakdown of fiber observed in looser stools is an indication of the presence of cellulase, the enzyme that breaks down cellulose, working more effectively. The product may also aid in the prevention of acidosis, a common digestive problem in ruminants.

In the ambient environment arena, the fact that the product contains oxygen and surfactants from the polysaccharides provides for additional benefits such as odor reduction, and it provides a shiny coat for the animals. The oxygen helps to reduce anaerobic conditions that produce malodors and produce conditions for flies to lay their eggs. Warts and fungal infections may be treated with the enriched oxygen.

In the barns where cows treated with the product are kept, especially where the product is applied, fly populations are also lower. It is contemplated that the aerobic environment created by the product discourages egg laying by the flies. In preferred embodiments, approximately 30-200 gallons have been applied per acre, depending on the tightness of the soil, how much is necessary to moist the surface for fly control, etc. . . . Preferably, the ground may be srayed to moistness, but not wetness. Moreover, outdoor pens and corrals have significantly better drainage when product is applied to the soil surface. Standing water during wet periods is reduced or eliminated because of better infiltration as described below.

For wastewater lagoon treatments, top water can be used by itself or mixed with micro-algae harvested from mature generators. It may be sprayed on the surface of the lagoon or pumped into the lagoon. The observed effects are enhanced bubbling from release of carbon dioxide and methane gases at depth, solids rising to the surface, and eventual liquefaction of all solid manure wastes in the lagoon. The lagoon becomes an extension of the animal's digestive system, producing more complete breakdown of feeds and feed residue and enhancing the transformation of nutrients into plant-available forms when the water is recycled back to croplands.

The activity of algae at the lagoon surface has several beneficial effects, including the absorption of carbon dioxide, and the raising of pH levels. Algae activity makes hydrogen sulfide and related compounds more soluble, thereby reducing odor. Oxygen is produced, therefore promoting an aerobic environment at the lagoon surface, even as the anaerobic environment is enhanced at depth.

Additionally, the lagoon is an extension of the animal's gut, but often contains overwhelming loads of undigested and partially digested materials. The disclosed system provides algae and enzymes to enhance the digestion of lagoon products, and by doing so, it prepares the nutrients to be more effectively used by higher plants in field crops. In the ideal, the lagoon becomes a “generator” and the “top water” can go to the field to perform nutrient exchange in the soil more effectively, as it does in the animal intestine.

It is contemplated that in the soil, polysaccharides are responsible for soil tilth, or crumb structure, and therefore directly related to nutrient flow, water holding capacity, and water flow through the soil. It is further contemplated that the product enhances nutrient breakdown, improves cation exchange capability, provides carbon in useful forms, provides micronutrients to enhanced plant health, etc. The product may also encourage nitrogen-fixing bacteria in the soil, as well as increasing the organic matter content in soils.

For field crops, the observed effects include better water penetration into the soil, better water retention, and higher crop yields. Top water, sometimes with addition of micro-algae, may be added to irrigation water or sprayed on the soil surface. In addition, observation has shown that salinity-damaged soils may be returned to productivity with treatment.

It is contemplated that the product of this disclosure may be used with animals other than ruminants. For example, horses may show positive results as disclosed above in relation to ruminants. While the horse is not a ruminant, it has microflora in the small intestine that does some of the same work as the rumen, including breakdown of cellulose, production of B vitamins, and the protection of intestinal mucus, and horses have shown positive indications using the products of this disclosure, as have dogs. It is contemplated that a wide variety of animals may be treated, including animals such as turkeys, chickens, pigs, and sheep.

While embodiments and applications of this invention have been shown and described, it will now be apparent to those skilled in the art having the benefit of this disclosure that many more modifications than mentioned above are possible without departing from the inventive concepts disclosed herein.

We claim:

1. A method for producing a micro-algae product comprising:
a. collecting urine from lactating cows;
b. mixing said collected urine with aerobically digested cow manure to form a mother liquor;
c. fermenting said mother liquor in an algae growth tank; and
d. forming two distinct layers of top water, including a top water layer, said top-water layer including yeast by-products and algae by-products.
